(19) United States
(12) Patent Application Publication
(10) Pub. No.: US 2020/0237626 A1
(43) Pub. Date: Jul. 30, 2020

(54) Anti-Aging Corrective and Protective Formulation and Methods

(71) Applicant: JAMRM, LLC, Dallas, TX (US)

(72) Inventors: Tatiana KELLY, Westlake, TX (US); Alisar Salah ZAHR, Dallas, TX (US); Rita Dixiana CABRERA, Ammon, ID (US); John KOCH, Ft. Worth, TX (US); Theresa FOX, The Colony, TX (US); Nirmal SHAH, Arlington, TX (US)

(21) Appl. No.: 16/259,600

(22) Filed: Jan. 28, 2019

(51) Int. Cl. A61K 8/04; A61K 36/9068; A61K 8/97; A61K 8/06; A61K 8/67; A61K 8/68; A61K 36/68

(52) U.S. Cl. CPC A61K 8/042; A61K 36/9068; A61K 8/97; A61K 8/062; A61K 8/064; A61K 36/68; A61K 8/671; A61K 8/678; A61K 8/673; A61K 8/68; A61K 8/066

(57) ABSTRACT

There are presented topical formulations and methods of applying these formulations in therapeutically effective amounts directly to areas of the human skin to remediate and to counter effects that are commonly regarded as aging. The formulations address the effects on the skin of UV light, High Energy Visible (HEV) light, blue light, and Urban Dust as well as remediating fine lines and age-related localized melanin concentrations (“age spots”), improving the uniformity of skin tone (reduction in skin redness) and enhancing the brightness of the skin to provide a more youthful appearance. Skin treated with the formulations also exhibit a reduction in the depth and prominence of wrinkles, a marked reduction in harmful reactive oxygen species in the treated skin, and a reduction in lipid peroxidation caused by Urban Dust.

### BACKGROUND

### 1. Field of the Invention

The inventions relate to formulations that treat effects of aging and environmental factors on the human skin, and more particularly formulations that are topically applied and have an anti-aging effect and that also provide an effect of protecting the skin and correcting existing damage of the skin.

### 2. Description of the Related Art

Human skin is a multi-layered tissue, often referred to as the “largest organ in the body.” It is complex, both in terms of structure, being multi-layered of varying porosity, and supplied with innumerable blood vessels, as well as in terms of its biochemistry and biome. Further complications are caused by factors in the surrounding environment that adversely affects the skin.

People have been aware of changes in the visible appearance and texture of human skin that take place over the years as a person ages. And, for generations people have attempted to use cosmetic, topically applied compositions to minimize these appearance and texture changes or to reverse these changes caused by age.

As people have studied skin change over time, it has become apparent that some of the changes in skin “age” is affected by, or accelerated by, the surrounding environment. For example, as sun bathing and tanning became more common, studies implicated ultra-violet radiation (from the sun or tanning beds) in premature skin aging and possibly in certain malignancies. It is now widely known that the skin is adversely affected by harmful ultra-violet light rays. There is now a growing body of evidence to support a suspicion that human skin is also affected by common artifacts of modern life such as electronic devices such as smart phones, laptops, computer monitors and TV screens that generate High Energy Visible (HEV) also known as “blue light,” which contributes to visible signs identified with skin aging. In addition, increasing urbanization and the ongoing use of fossil fuels contribute to an increase of minute particulate matter, commonly referred to as “Urban Dust,” which is also believed to affect human skin causing visible signs identified with skin aging.

### SUMMARY

There are presented topical formulations and methods of applying these formulations in therapeutically effective amounts directly to areas of the human skin to remediate and to counter effects that are commonly regarded as aging. Consequently, the formulations may be referred to as “anti-aging formulations.”

In general, the anti-aging formulations address the effects on the skin of UV light, visible blue light and Urban Dust. Moreover, the formulations address and remediate fine lines and age-related localized melanin concentrations (“age spots”), improves the uniformity of skin tone (reduction in skin redness) and enhances the brightness of the skin to provide a more youthful appearance. Skin treated with the formulations also exhibit a reduction in the depth and prominence of wrinkles. Further, the anti-aging formulations produce a marked reduction in harmful reactive oxygen species in the treated skin and a reduction in lipid peroxidation caused by Urban Dust.

In an exemplary embodiment there is provided a formulation A topical skin care formulation having therein at least a stabilized functionalized form of ascorbic acid that releases ascorbic acid when applied to the skin; a deglycosilated isothiocyanate; a phenylpropanoid; and an acetyl zingerone; where the components are commingled in a base comprising an emulsion of oil-in-water or water-in-oil.

In an exemplary method of applying the topical formulations to human skin to remediate effects of aging, the method including at least the steps of:

topically applying a formulation having components that include but are not limited to:

a stabilized functionalized form of ascorbic acid that releases ascorbic acid when applied to the skin; a deglycosilated isothiocyanate; a phenylpropanoid; and acetyl zingerone;

where the components are commingled in a base comprising an emulsion of oil-in-water or water-in-oil; and repeating the step of topically applying the formulation twice per day to deliver a therapeutically effective dose to the skin.

The foregoing provides a brief non-comprehensive summary of the inventive formulations and methods that are presented in more detain here below.

### BRIEF DESCRIPTION OF THE DRAWINGS

The foregoing aspects and many of the attendant advantages, of the present technology will become more readily appreciated by reference to the following Detailed Description, when taken in conjunction with the accompanying simplified drawings of exemplary embodiments. The drawings, briefly described here below, are not to scale, are presented for ease of explanation and do not limit the scope of the inventions recited in the accompanying patent claims.

FIG. 1 illustrates by bar chart the decrease in melanin content (“brightening”) of skin achieved by treatment with an exemplary embodiment of the inventive formulation or composition as compared to untreated skin, and as against a comparative formulation.

FIG. 2 is a color photo which illustrates the upregulation of intracellular glutathione of skin cell HaCaT keratinocytes after 24 hours of incubation with treatment.

FIG. 3 illustrates by bar chart the protection afforded against reactive oxygen species by treatment with an exemplary inventive formulation versus a comparative formulation at 30, 60, and 120 minutes exposure to high energy visible light (blue light).

FIG. 4A illustrates by bar chart the performance of an exemplary embodiment of the inventive formulation in the reduction of reactive oxygen content in human skin as compared to non-exposed skin, and as compared to exposed skin treated with other formulations.

FIG. 4B illustrates by bar chart the performance of an exemplary embodiment of the inventive formulation in the lipid peroxidation content in human skin as compared to non-exposed skin, untreated skin, and as compared to exposed skin treated with another formulation.

FIG. 5A is a color photo showing a baseline of human facial skin near an outer corner of an eye where melanin is shown in red.

FIG. 5B is a color photo of the same area of human facial skin as in FIG. 5A showing a reduction in melanin after treatment with an exemplary embodiment of the formulation, in an exemplary treatment protocol.

FIG. 6 illustrates in bar chart form the performance of an exemplary embodiment of the inventive formulation after 4 weeks of treatment versus a comparative formulation in terms of radiance, skin smoothness, wrinkles, fine lines (crow's feet type), and global facial fine lines.

FIG. 7A is a color photo showing human skin and depicting the depth of wrinkles in the skin by the dark blue-green markings that correlate to the scale accompanying the figure.

FIG. 7B is a color photo showing the same human skin as in FIG. 2A after a treatment protocol with applications of an exemplary embodiment of the inventive formulations, depicting the reduction in the depth of wrinkles in the skin evidenced by a reduction in the area of the dark blue-green markings.

FIG. 8 is a graph representing a decrease from baseline of global facial skin tone redness (and improvement in skin tone uniformity) in persons being treated with topical application of an exemplary embodiment of the inventive formulation, at weeks 4, 8 and 12 of treatment.

### DETAILED DESCRIPTION

The patent or application file contains at least one drawing executed in color. Copies of this patent or patent application publication with color drawing(s) will be provided by the Office upon request and payment of the necessary fee.

The following non-limiting detailed descriptions of examples of embodiments of the invention may refer to appended Figure drawings and are not limited to the drawings, which are merely presented for enhancing explanations of features of the technology. In addition, the detailed descriptions may refer to particular terms of art, some of which are defined herein, as appropriate and as necessary for clarity.

There are presented topical formulations for human skin, especially facial skin, and methods of applying these formulations in therapeutically effective amounts directly to areas of the human skin to remediate, counter and provide protection against effects that are commonly regarded or associated with aging of the person and his/her skin. Consequently, taken in this context, the formulations may be referred to as “anti-aging formulations.”

In general, the formulations address several effects on the skin over time, during the lifetime of the person, and also address environmental issues that impact the skin, such as but not limited to dwelling in an urban environment and exposure to fine particulate matter in the air, or working in an office environment with exposure to light emitting electronic devices, for example computer monitors, and the like. Thus, the anti-aging formulations address the effects of UV (ultra violet) light, visible blue light (400-700 nm), HEV light (blue light of 400-450 nm) and Urban Dust. Moreover, the formulations also address and remediate fine lines, such as crow's feet for example, and age-related localized melanin concentrations (“age spots”). The anti-aging formulations also enhance the brightness of the skin by, for example, a reduction of localized areas of melanin concentration in the skin to provide a more youthful appearance. The anti-aging formulations improves the uniformity of skin tone by a reduction in skin redness and making the tone more uniform, or even. Skin treated with the formulations also exhibit a reduction in the depth and prominence of wrinkles. Further, the formulations produce a marked reduction in harmful reactive oxygen species in the treated skin caused by Urban Dust; and a reduction in lipid peroxidation caused by Urban Dust.

The anti-aging formulations are topically applied to the skin, preferably the facial area that is affected by the signs of aging that is desired to be addressed. Depending upon the formulation, which might vary in terms of the active ingredient concentrations, the anti-aging formulation may be conveniently applied twice daily: in the morning and in the evening.

The formulation may be in the form of a lotion, a cream, or a gel, depending upon the base carrier composition. The base carrier may be a water-in-oil emulsion, or an oil-in-water emulsion. The oil may be, but is not limited to, silicone oil. The base carrier could also be anhydrous. Since the base acts as a carrier, it ought to be non-reactive with the ingredients of the formulation, and it ought to allow penetration of the ingredients into the skin, as appropriate, for therapeutic purposes.

Ingredients of the exemplary embodiments of the formulations may be divided into those that are primarily regarded as corrective ingredients (i.e. those that remediate existing skin damage) and those that are primarily regarded as protective ingredients (i.e. those that protect from potential harm). While these labels may be used herein, they should not be applied strictly since some ingredients may fall into both corrective and protective categories, and the primary function might vary.

Exemplary primarily corrective ingredients of the anti-aging formulation include Tetrahexyldecyl Ascorbate (THD ascorbate), Hydrolyzed Eruca Sativa Leaf, Plantago Lanceolate Leaf Extract, and Sodium Carboxymethyl beta glucan, and equivalents or functionalized versions of these. An exemplary primarily protective ingredient includes: Acetyl Zingerone, THD Ascorbate, Punica Granatum Extract, and Ergothioneine.

The anti-aging formulations include THD ascorbate, which is a preferred stabilized, functionalized form of ascorbic acid, but not the only such form. Due to the lipophilic nature of the skin and the hydrophilicity of ascorbic acid itself, delivery of it to the skin and penetration into the skin is impeded. The use of a stabilized, functionalized form of ascorbic acid such as THD ascorbate ensures delivery of the molecule to the desired skin site where it can then release ascorbic acid into the skin. THD Ascorbate reduces melanin in the skin thereby reducing visible age spots. THD Ascorbate also promotes the production of collagen in the skin resulting in reduction in the depth of wrinkles and smoothing out of fine lines. THD Ascorbate also decreases or shields from the effects of UV-A and UV-B radiation.

Exemplary embodiments of the anti-aging formulations include hydrolyzed Eruca Sativa leaf. This is an extract of the leafy vegetable, arugula, and it is high in natural deglycosilated isothiocyanate concentration. Deglycosilated isothiocyanate is an active corrective ingredient that promotes the skin's own production of the antioxidant peptide, glutathione (smart antioxidant technology). Glutathione is high in sulfur and has an odor that inhibits its use in topically applied products. Thus, use of this ingredient presents new technology that allows the skin to boost its own levels of glutathione production. Glutathione is a powerful antioxidant that in addition also assists in the natural regeneration of Vitamins C and E in the skin, while providing skin soothing and calming benefits.

Another corrective ingredient in exemplary embodiments of the anti-aging formulation is Plantago Lanceolata leaf extract which has strong antioxidant properties, containing a high concentration of the antioxidant plantamajoside. Plantago Lanceolata leaf extract is a source of phenylpropanoid. This extract enhances collagen I and elastin synthesis and enhances the synthesis of collagen IV and laminins at the dermal-epidermal junction (DEJ). In addition, it inhibits the formation of harmful Matrix Metalloproteinases (MMPs). These MMPs are enzymes activated by external factors such as, UV light exposure, or by intrinsic factors such as inflammation, MMPs have the undesirable effect of contributing to the breakdown of collagen while inhibiting new collagen formation. Thus, they are implicated in fine line and wrinkle formation. Moreover, Plantago Lanceolata leaf extract brightens and evens skin tone, especially in the presence of high energy blue light (“HEV”). Melanin synthesis from HEV blue light is persistent, and this blue light is ubiquitous: the sun and electronic device screens emit HEV blue light. Further, Plantago Lanceolata leaf extract aids in the reduction of damage from prolonged exposure to light energy emitted from electronic device screens, while also increasing skin density and firmness while reducing inflammation.

Another corrective ingredient in exemplary embodiments of the anti-aging formulation is Euterpe Oleracea fruit extract, commonly known as Açai. This ingredient has strong antioxidant properties. More particularly, it contains a high concentration of the antioxidant, ellagic acid. It assists in the brightening of skin through tryosinase inhibition. Further, it also inhibits harmful MMP formation.

An optional ingredient in exemplary embodiments of the anti-aging formulation is Punica Granatum Extract, commonly known as pomegranate extract. This ingredient has strong antioxidant properties, containing a high concentration of the antioxidant, ellagic acid. It brightens skin through tyrosinase inhibition. And, in addition, it inhibits formation of MMPs.

Another corrective ingredient in exemplary embodiments of the anti-aging formulation is acetyl zingerone. Acetyl zingerone assists in maintaining the integrity of skin lipids. This is a significant function because natural lipids present in the skin can degrade and promote inflammation. It also prevents DNA damage by reducing the immediate formation of cyclobutane pyrimidine dimers (“CPDs” implicated in skin cancers) in the presence of UVA radiation and the delayed formation in the presence of UVB radiation). It also operates on free radicals by 2 different mechanisms of action: it reduces free radical generation and it neutralizes existing free radicals. Finally, acetyl zingerone also inhibits production of MMPs.

Another ingredient in exemplary embodiments of the anti-aging formulation is Diglucosyl Gallic acid. Diglucosyl gallic acid is converted into gallic acid, a trihydroxy benzoic acid, an antioxidant and a natural inhibitor of tyrosinase (enzyme required in the melanogenesis process). The conversion of Diglucosyl gallic acid into gallic acid takes place on the skin surface mediated by enzymes (glucosidases) secreted by the microorganisms found in the skin microbiome. Gallic acid blocks melanogenesis process by controlling the expression of MITF a gene required in the melanogenesis process, stops melanin transfer by saturating keratinocytes receptors, and blocks melanin synthesis even under UV conditions.

Another optional ingredient in exemplary embodiments of the anti-aging formulation is sodium carboxymethyl beta glucan. This is a polysaccharide derived from Baker's Yeast (Saccharomyces Cerevisiae) that reduces skin irritation. It also enhances the skin's barrier function and increases the skin's moisture levels. It protects from lipid oxidation in the presence of UVA radiation. And sodium carboxymethyl beta glucan reduces fines lines and wrinkles.

Another ingredient in exemplary embodiments of the anti-aging formulation is palmitoyl glycine which is naturally found in the brain and the skin. It is a compound that uses amino acid technology to reduce fine lines and wrinkles, reduce redness, and reduce production of MMPs. The ingredient enhances luminosity and brightness.

Another ingredient in exemplary embodiments of the anti-aging formulation is ergothioneine, which is often synthetic, but bio-identical to that found in nature (e.g. in mushrooms). This ingredient enhances ATP synthesis for increased cellular energy levels. It operates synergistically with THD Ascorbate and scavenges the superoxide anion and singlet oxygen radical.

In an exemplary embodiment, the anti-aging formulation includes: Tetrahexyldecyl Ascorbate; acetyl zingerone; a phenylpropanoid, and a deglycosilated isothiocyanate in a base carrier of a water-in-oil emulsion, an oil-in-water emulsion, a gel or an anhydrous carrier.

In another exemplary embodiment, the anti-aging formulation includes by weight percent based on the total weight of the formulation: from about 3 wt. % to about 60 wt. % or from about 10 wt. % to about 60 wt. %, or from about 3 to about 10 wt. % THD Ascorbate; commingled with acetyl zingerone; a phenylpropanoid; and a deglycosilated isothiocyanate in a base carrier of a water-in-oil emulsion, an oil-in-water emulsion, a gel or an anhydrous carrier.

In another exemplary embodiment, the anti-aging formulation includes by weight percent based on the total weight of the formulation: from about 3 wt. % to about 60 wt. % or from about 10 wt. % to about 60 wt. % or from about 3 wt. % to 10 wt. % THD Ascorbate, commingled with from about 0.1 to about 6 wt. % acetyl zingerone; from about 0.005 to about 0.06 wt. % deglycosilated isothiocyanate; about 0.001 to about 0.02 wt. % phenylpropanoid, preferably but not necessarily, derived from Plantago Lanceolata leaf extract; and sodium carboxymethyl gluconate, all in a base carrier of a water-in-oil emulsion, an oil-in-water emulsion, a gel or an anhydrous carrier.

The exemplary embodiments of the anti-aging topical skin care formulation may also include one or more of the peptides, including but not limited to: Palmitoyl Tripeptide-5, Palmitoyl Tripeptide-38, Palmitoyl Dipeptide-5 Diaminobutyroyl Hydroxythreonine, Tetradecyl Aminobutyroylvalylaminobutyric Urea Trifluoroacetate, Acetyl Tetrapeptide-2, Trifluoroacetyl Tripeptide-2, and Palmitoyl Hexapeptide-12.

Exemplary embodiments of the anti-aging formulations may also include one or more of pentylene glycol, dimethyl sulfone, urea, tetrahydropiperine, and lipophilic molecules including cholesterol or isopropyl myristate as a skin penetration enhancer.

Further, exemplary embodiments may also include ingredients such as vitamins A, B, D and E. In addition, embodiments may include ceramides and growth factors, as appropriate.

Exemplary embodiments of the formulation may include one or both of glucans and glucosides, as appropriate.

In addition, exemplary embodiments of the anti-aging formulation may include growth factors, including but not limited to: KGF (Keratinocyte growth factor: Palifermin CAS #162394-19-6); TGFβ (Transforming Growth Factor beta CAS #122304-04-5); VEGF (Vascular Endothelial Growth Factor Receptor-3 CAS #144638-77-7) and PDGF (Platelet Derived Growth Factor Platelet-activating factor [1-Hexadecyl-2-acetyl-glycero-3-phosphocholine CAS #74389-68-7] Platelet activating factor phosphatidate CAS #65154-06-5 [1-O-Alkyl-2-acetyl-sn-glyceryl-3-phosphorylcholine] Platelet Factor 1 CAS #62031-46-3, Platelet Factor 3 CAS #37270-93-2, or Platelet Factor 4 CAS #37270-94-3)

Exemplary embodiments of the formulation exclude compositions that are known to be toxic, or allergenic, and that are harmful to human skin.

The following examples are non-limiting and serve to illustrate the effectiveness of the inventive anti-aging formulations when used topically in therapeutic amounts at intervals, as described herein, and as may be prescribed.

### EXAMPLES

### Example 1

Tests were conducted in vitro using a MelanoDerm™ 3D tissue assay to determine whether the inventive anti-aging formulation reduces melanin concentration in skin tissue. This comparative test was conducted against a test serum that included THD ascorbate at 30 wt. %. The assay was a 14-day test in which every 24 hours, the tissues were rinsed with Phosphate Buffered Saline (PBS) solution and fresh material (test serum or the inventive anti-aging formulation) was applied and the media was changed as well. Melanin content was determined after 14 days with tissues homogenized and digested. Tissue melanin content were determined as μg melanin per tissue.

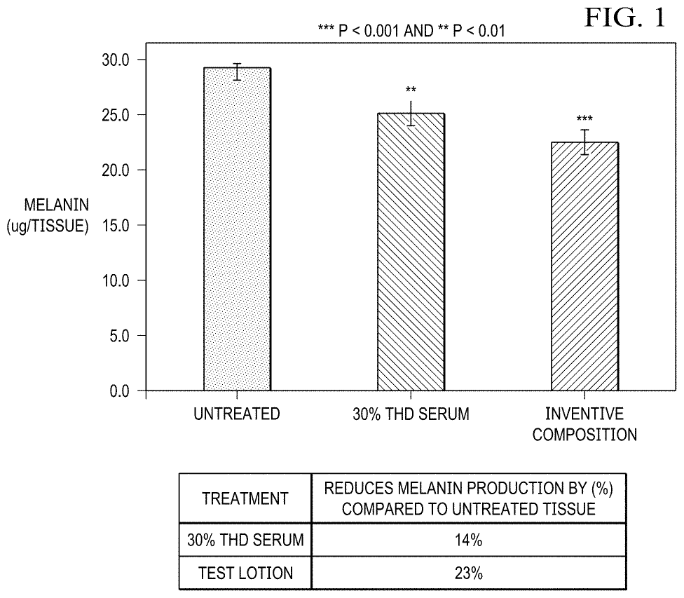

FIG. 1 shows the results in a bar graph. In the untreated tissue, the melanin increased to nearly 30 μg/tissue. In the case of the tissue treated with a comparative test serum containing THD ascorbate, the melanin was at about 26 μg/tissue. In the case of the tissue treated with the exemplary anti-aging formulation of the invention, the melanin was at about 23 μg/tissue.

Accordingly, the inventive anti-aging formulation has superior melanin reducing properties as compared to the comparative THD ascorbate containing serum, and also as compared to the baseline.

### Example 2

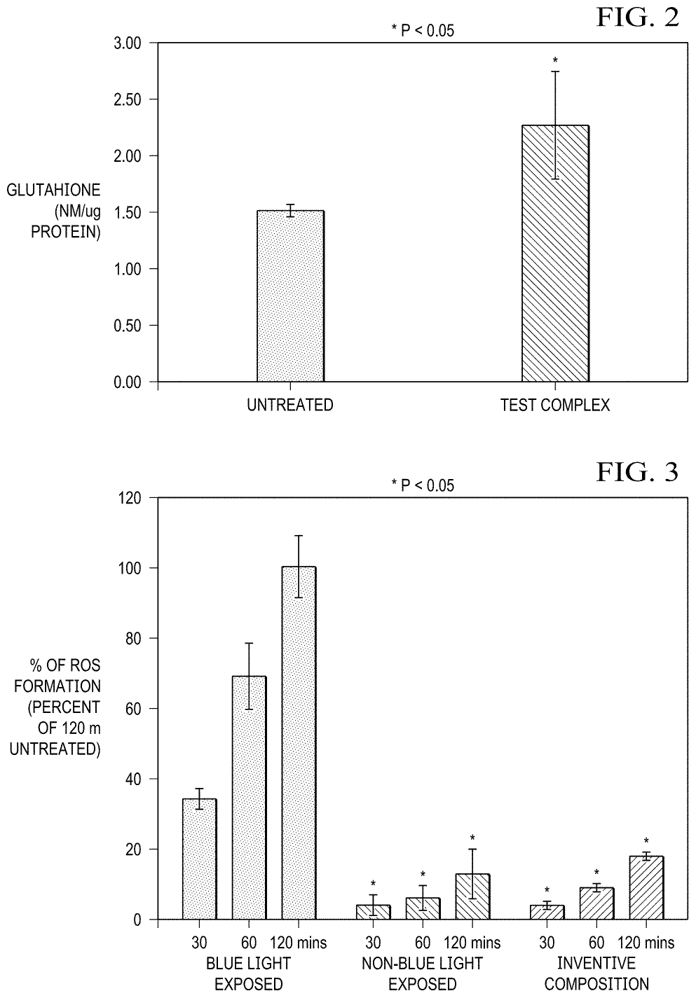

Tests were conducted in vitro to assess the levels of glutathione, a skin lightener, in samples treated with an exemplary embodiment of the inventive formulation versus untreated samples. Intracellular levels of small molecular weight antioxidant glutathione were determined after 24 hours of incubation with the exemplary embodiment of the anti-aging formulation against a negative control (untreated) in HaCaT keratinocytes. FIG. 2 shows levels of glutathione have been upregulated by 49% after 24 hours, from 1.51 (nM/μg Proteins) to 2.25 (nM/μg Proteins).

### Example 3

Tests were conducted to determine a degree of protection provided against reactive oxygen species (ROS) produced by High Energy Visible (HEV) light also known as Blue Light in the 450 nm range. These tests were conducted in vitro, using the MatTek™ EpiDerm™ tissues.

Exposure to blue light, causes skin aging effects including prolonged hyperpigmentation, melanin synthesis, and creation of free radicals such as ROS. Accordingly, measuring the amount of reactive oxygen species formed and determining whether the anti-aging formulation protects against the formation of ROS in vitro is highly probative of its efficacy in vivo.

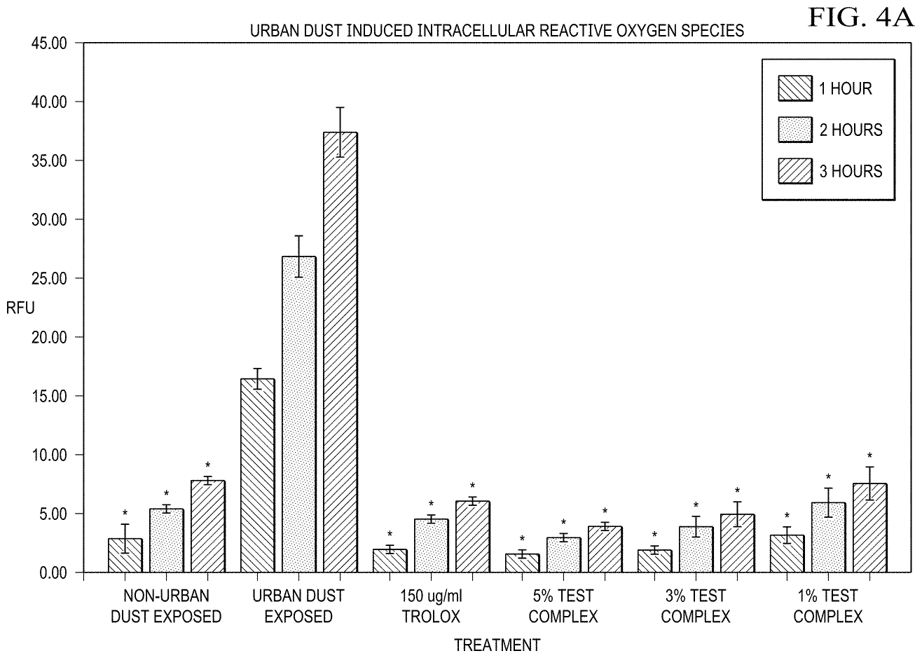

FIG. 3 has bar graphs that illustrate the percent of reactive oxygen species (ROS) formed when exposed to blue light over periods of 30, 60 and 120 minutes. The sample treated with the exemplary anti-aging formulation approximates closely to the sample not at all exposed to blue light in terms of % of reactive oxygen species. This test confirms the efficacy of the inventive anti-aging formulation in preventing reactive oxygen species formation and avoiding resultant damage to the skin.

### Example 4

A third-party testing facility conducted tests to determine the effects of standardized Urban Dust particulates (NIST® SRM® 1649b) on the production of free radicals and lipid peroxidation in human skin cells (HEKn keratinocytes). Tests were conducted at 1 hour, 2 hours and 3 hours with varying concentrations of the test samples (1%, 3%, and 5%) (i.e. at one hour intervals) to measure intracellular reactive oxygen species and after 4 hours with test sample (5%) for lipid peroxidation. A fluorescent plate reader was utilized to measure a fluorescent dye incubated in all test conditions which measures intracellular reactive oxygen species and lipid peroxidation. The measurement is expressed as the mean relative fluorescent unit (RFU).

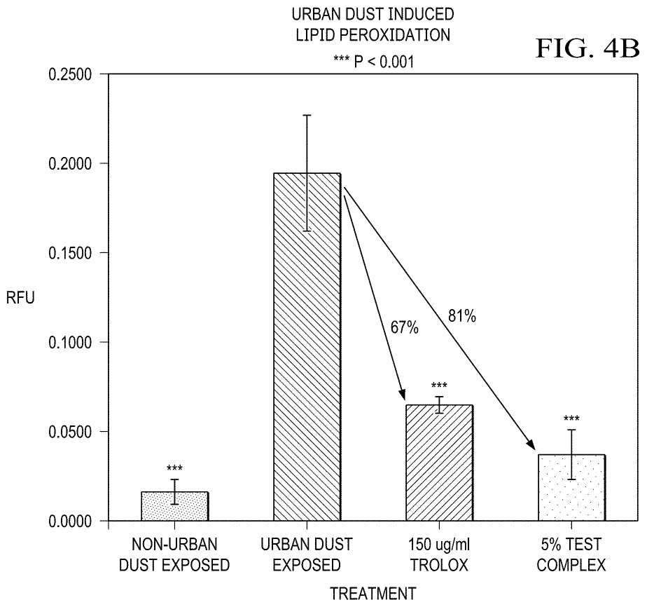

FIG. 4A. As one might expect, the samples not exposed to the standardized Urban Dust had very low RFU. Those exposed to the Urban Dust had high RFU that increased over time. Those samples treated with a control had RFU comparable to those that were untreated. Three samples were treated with doses of the inventive anti-aging formulation at 1%, 3% and 5% and were exposed to the standardized Urban Dust. At 3 and 5% these showed RFU values better than the samples that were not treated with Urban Dust. The 1% sample matched the RFU of the untreated samples.

These tests demonstrate the efficacy of the inventive anti-aging formulations in protecting against free radicals induced by Urban Dust. Tests were conducted to determine the effects of Urban Dust on lipid peroxidation in vitro.

FIG. 4B illustrates by bar graph the effects of the Urban Dust on tissue. Non-exposed tissue has immeasurably low RFU. The urban dust exposed cells have an increase in lipid peroxidation with 0.2 RFU. The sample treated with a control (Trolox) and exposed to the Urban Dust had an RFU of about 0.05 with a 67% reduction in lipid peroxidation. The tissue treated with the inventive anti-aging formulation and exposed to the Urban Dust had an RFU of about 0.025 with an about 80% (81) reduction in lipid peroxidation. These tests demonstrate the efficacy of the inventive anti-aging formulations in protecting against lipid peroxidation induced by Urban Dust.

### Example 5

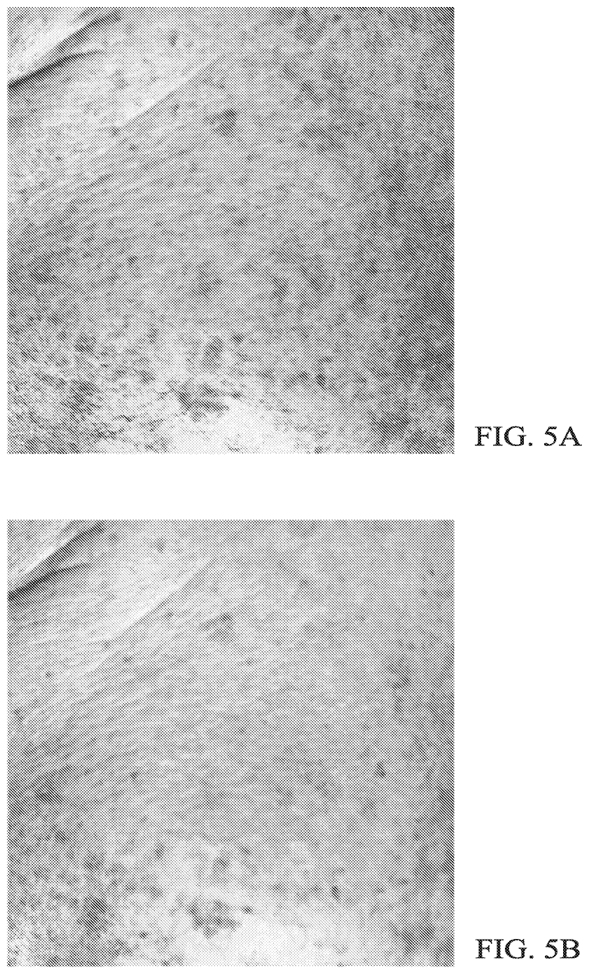

A selected subject was photographed with an Antera 3D® (Miravex Limited, Dublin, Ireland) camera . FIG. 5A is a photograph taken as a “baseline” before the subject had undergone any facial treatment. The photo depicts an area on the outside corner of an eye and shows significant amounts of red, orange, and light mottled areas or spots. The red represents areas of higher melanin content or intensity in the skin—also known as “age spots.”

FIG. 5B is a photograph taken after 12 weeks of treatment with an exemplary embodiment of the anti-aging formulation on 31 subjects ages 38-60 years with twice daily use. There is a clear decrease in the red areas in the same corner of the eye region. The subject had been treated with the application of an exemplary embodiment of the anti-aging formulation twice daily, applied to the face and to the depicted facial region. Data analysis indicates 88% of subjects showed an improvement in melanin reduction at 8 weeks. The photos attest to the efficacy of the anti-aging formulation in removing melanin and age spots.

### Example 6

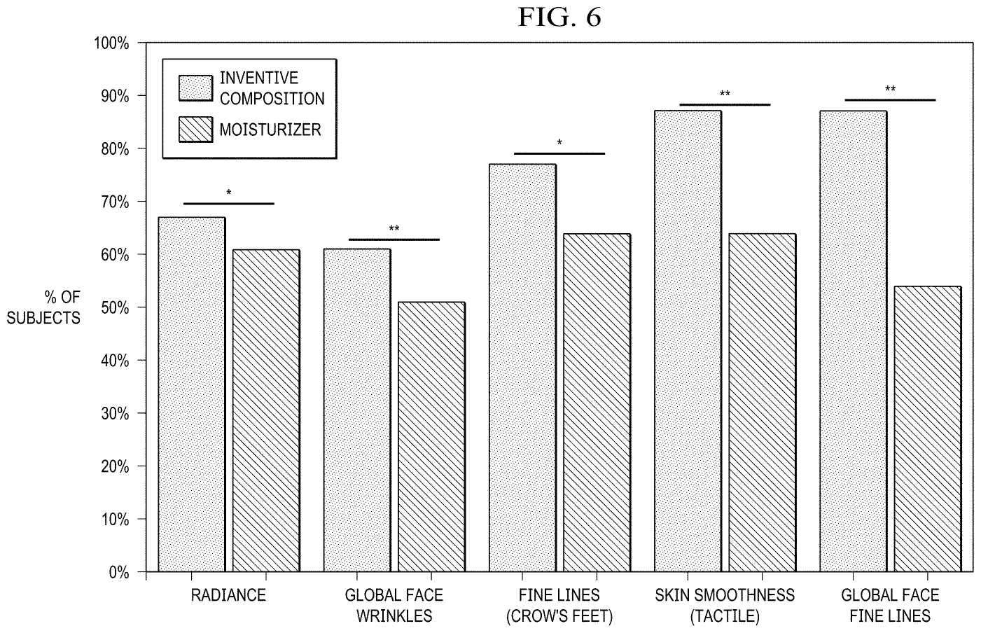

A comparative test of an exemplary embodiment of the anti-aging formulation against a moisturizer that was the baseline was conducted in a double blind, single center study. Tests were conducted over 12 weeks during which time an exemplary embodiment of the anti-aging formulation was applied twice daily to the facial areas of each of the subjects in both treatment groups. Efficacy parameters radiance, skin smoothness (tactile), global face wrinkles, fine lines (Crow's feet), and global face fine lines were measured by a clinical grader using the modified Griffith 10-point scale according to the numerical definitions; 0 indicating none (best possible condition), 1 to 3 indicating mild, 4 to 6 indicating moderate, and 7 to 9 indication severe (worst possible condition). The percent of subject improvement was calculated against baseline values. Results are summarized in bar chart format in FIG. 6 that depicts results for 31 subjects, ages 38-60 years at the 4-week time point. Superior performance of the exemplary embodiment of the anti-aging formulation is demonstrated.

Radiance or brightness is better in about 70% of the subjects using the anti-aging formulation as compared to 60% of the subjects using the moisturizer.

Skin smoothness (tactile) was superior in the case of in about 90% of the subjects using the anti-aging formulation as compared to 60% of those using moisturizer.

Global facial wrinkles were improved in about 60% of subjects using the anti-aging formulation as compared to about 50% using the moisturizer.

Fine lines (Crow's Feet) were improved in about 75% of subjects using the anti-aging formulation as compared to about 63% using the moisturizer.

Global facial fine lines were improved in about 87% of subjects using the anti-aging formulation as compared to about 53% using the moisturizer.

The results clearly demonstrate the superiority of the anti-aging formulation over a moisturizer.

### Example 7

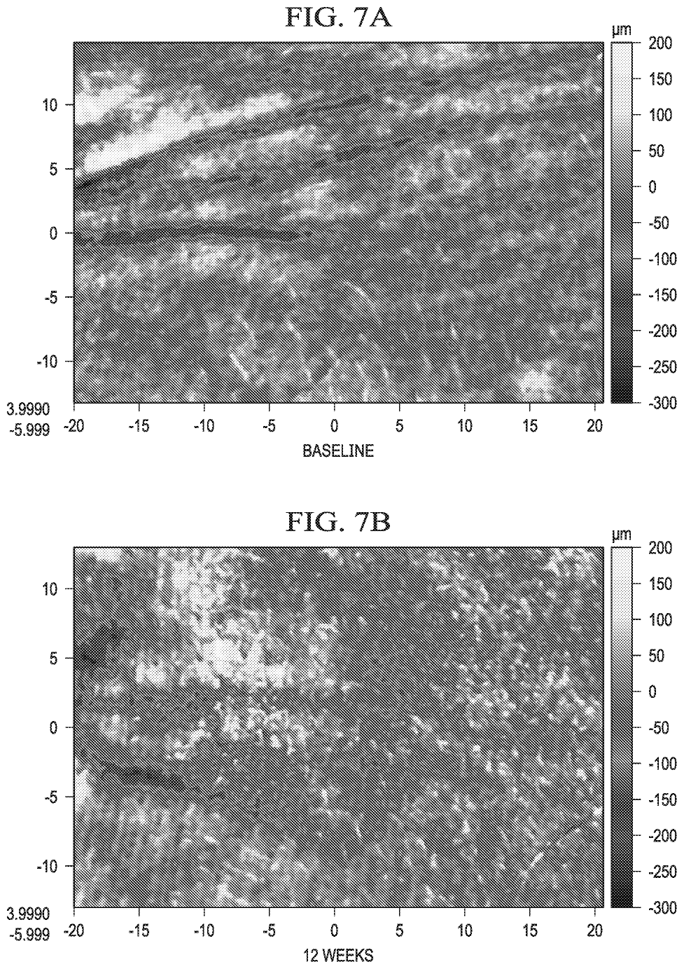

PRIMOS Lite 45×30 mm system is a handheld 3D imaging device used to assess the microtopography of the skin. Wrinkle analysis was performed, and measurements of wrinkle depth, volume, length, area, and count were taken. FIG. 7A is a clinical photo taken with PRIMOS Lite of an area near the corner of a subject's eye. The camera can detect depth of the wrinkles on the skin, and the depth is shown on a scale alongside FIGS. 7A and 7B. There were 31 subjects in the test ages 38-60. First, to establish a baseline, photos were taken to establish the wrinkle depth at the crow's feet region near the eye. A sample of this is shown for a subject in FIG. 7A.

After twelve weeks of treatment by application of an exemplary embodiment of the anti-aging formulation twice daily, photos were taken of the same region again. FIG. 7B shows the change in wrinkle depth for the same subject of FIG. 2A. The dark blue area showing the deepest wrinkle region is significantly reduced. Thus, the appearance of the subject is that of being more youthful. It was found that after 12 weeks of twice daily use with the exemplary embodiment of the anti-aging formulation, 77% of clinical study participants showed a decrease in wrinkle volume and 73% of clinical study participants showed a decrease in wrinkle depth. Furthermore, the average reduction of 10% in wrinkle volume and wrinkle depth was determined after 12 weeks of twice-daily use.

### Example 8

Tests were conducted to measure the efficacy of the inventive anti-aging formulation in providing skin tone evenness, which is both a reduction in redness of the skin, as well as skin tone uniformity.

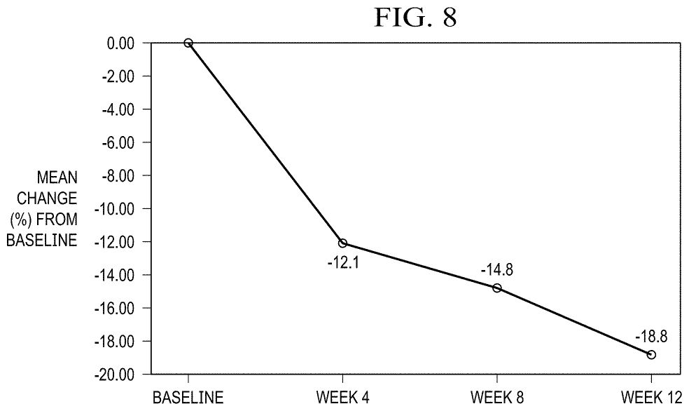

A number of subjects (31) underwent treatment with an embodiment of the anti-aging formulation which was topically applied twice daily to the facial area. Measurements were taken every 4 weeks by a clinical grader utilizing the modified Griffith 10-point scale. The parameter that was evaluated by the clinical grader was skin tone evenness with emphasis on redness. A measurement of 0 indicates even, healthy skin color and a measurement of 9 indicates uneven, discolored appearance. At each time point, the percent of improvement or worsening of this efficacy parameter as well as the percent mean change from baseline was calculated. FIG. 8 shows a steep decline in redness by 12 percent after 4 weeks, almost 15% after 8 weeks, and almost 20% after 12 weeks.

These tests establish the efficacy of the formulation in treating for skin tone evenness.

While examples of embodiments of the technology have been presented and described in text, and some examples also by way of illustration, it will be appreciated that various changes and modifications may be made in the described technology without departing from the scope of the inventions, which are set forth in and only limited by the scope of the appended patent claims, as properly interpreted and construed.

1. A topical skin care formulation comprising, commingled in a base:
a stabilized functionalized form of ascorbic acid that releases ascorbic acid when applied to the skin;
a deglycosilated isothiocyanate;
a phenylpropanoid; and
acetyl zingerone.

2. The topical skin care formulation of claim 1, wherein the base is an oil-in-water base or a water-in-oil base.

3. The topical skin care formulation of claim 1, wherein the base is water-in-silicone or silicone-in-water.

4. The topical skin care formulation of claim 1, wherein the base is a gel.

5. The topical skin care formulation of claim 1, wherein the stabilized functionalized form of ascorbic acid comprises THD Ascorbate, and wherein the THD Ascorbate comprises from about 3 wt. % to 60 wt. % of the formulation.

6. The topical skin care formulation of claim 1, wherein the stabilized functionalized form of ascorbic acid comprises THD Ascorbate, and wherein the THD Ascorbate comprises from about 10 wt. % to 60 wt. % of the formulation.

7. The topical skin care formulation of claim 1, wherein the stabilized functionalized form of ascorbic acid comprises THD Ascorbate, and wherein the THD Ascorbate comprises from about 3.0 wt. % to 10 wt. % of the formulation.

8. The topical skin care formulation of claim 1, wherein the formulation comprises from about 0.1 to about 6 wt. % acetyl zingerone.

9. The topical skin care formulation of claim 1, wherein the formulation comprises from about 0.001 to about 0.02 wt. % of the phenylpropanoid.

10. The topical skin care formulation of claim 1, wherein the formulation comprises from about 0.005 to about 0.06 wt. % of the deglycosilated isothiocyanate.

11. The topical skin care formulation of claim 1, further comprising one more of the skin growth factors: VEGF, TGFβ, KGF, and PDGF.

12. The topical skin care formulation of claim 1, further comprising one or more of vitamins A, E, and B.

13. The topical skin care formulation of claim 1, further comprising peptides.

14. The topical skin care formulation of claim 1, further comprising one or more of the peptides: Palmitoyl Tripeptide-5, Palmitoyl Tripeptide-38, Palmitoyl Dipeptide-5 Diaminobutyroyl Hydroxythreonine, Tetradecyl Aminobutyroylvalylaminobutyric Urea Trifluoroacetate, Acetyl Tetrapeptide-2, Trifluoroacetyl Tripeptide-2, and Palmitoyl Hexapeptide-12.

15. The topical skin care formulation of claim 1, further comprising ceramides.

16. The topical skin care formulation of claim 1, further comprising a skin penetration enhancer.

17. The topical skin care formulation of claim 1, further comprising one or more of pentylene glycol, dimethyl sulfone, urea, tetrahydropiperine, and lipophilic molecules.

18. The topical skin care formulation of claim 17, wherein the lipophilic molecules of the formulation comprises one or more of cholesterol or low molecular weight esters; or one or more porosity ester oils as a skin penetration enhancer.

19. The topical skin care formulation of claim 17, wherein the lipophilic molecules of the formulation comprises one or more of isopropyl myristate and isopropyl palmitate.

20. The topical skin care formulation of claim 17, wherein the lipophilic molecules of the formulation comprises one or more of nonyl isononanoate and isodecyl isononanoate.

21. The topical skin care formulation of claim 1, further comprising one of or a mixture of glucans and glucosides.

22. The topical skin care formulation of claim 1, wherein the deglycosilated isothiocyanates are sourced from extracts of crucifers that include deglycosilated isothiocyanates.

23. The topical skin care formulation of claim 1, wherein the phenylpropanoids are sourced from an extract of Plantago Lanceolata Leaf.

24. A method of treating human skin for effects of aging, the method comprising the steps of:
topically applying the formulation of claim; and
optionally repeating the step of topically applying the formulation in order to deliver a therapeutically effective dose to the skin.

25. The method of claim 24, wherein the method reduces melanin formation up to 88% at 8 weeks of twice daily application of treatment.

26. The method of claim 24, wherein the method upregulates intracellular glutathione by 49% over 24 hours.

27. The method of claim 24, wherein the method reduces a reactive oxygen species in the skin caused by high energy blue light by about 90% during an hour of light exposure.

28. The method of claim 24, wherein the method reduces a reactive oxygen species in the skin caused by Urban Dust by about 90% within an hour.

29. The method of claim 24, wherein the method reduces lipid peroxidation in the skin caused by Urban Dust by about 80%.

30. The method of claim 24, wherein the method over a period of about 4 weeks reduces fine lines.

31. The method of claim 24, wherein the method reduces skin redness after about 4 weeks with continuing skin redness reduction for a period of 12 weeks.

32. The method of claim 24, wherein the method reduces skin redness by about 12% after about 4 weeks with continuing skin redness reduction of up to 18% at 12 weeks of treatment.
